(19) United States
(12) Patent Application Publication
(10) Pub. No.: US 2015/0140007 A1
(43) Pub. Date: May 21, 2015

(54) PHARMACEUTICAL COMPOSITION FOR TREATING AND/OR PREVENTING TYPE I DIABETES AND APPLICATION THEREOF

(71) Applicant: Beijing Advanccine Biotechnology Co., Ltd,, Beijing (CN)

(72) Inventors: Bin Wang, Beijing (CN); Guoxing Zheng, Beijing (CN); Shuang Geng, Beijing (CN); Yizhong Wang, Beijing (CN); Qingling Yu, Beijing (CN)

(21) Appl. No.: 14/394,051

(22) PCT Filed: Apr. 12, 2013
(86) PCT No.: PCT/CN2013/000429

§ 371 Date: Dec. 8, 2014

(30) Foreign Application Priority Data
Apr. 13, 2012 (CN) 201210108159.6

(51) Int. Cl. A61K 39/395; A61K 38/51; A61K 38/13; A61K 31/573; A61K 31/436; A61K 38/28; A61K 38/22

(52) U.S. Cl. CPC A61K 39/39541; A61K 38/28; A61K 38/51; A61K 38/22; A61K 31/573; A61K 31/436; A61K 38/13; A61K 2039/505
USPC 424/143.1; 424/184.1; 424/198.1; 424/185.1

(57) ABSTRACT

The present invention provides a composition for treating and/or preventing type I diabetes and an application thereof. The active ingredient of the composition is 1.) or 2.) or 3.), as follows: 1.) a mixture of a type I diabetes protein antigen and an immunosuppressor, 2.) a mixture of a type I diabetes protein antigenic epitope polypeptide and an immunosuppressor, 3.) a mixture of a type I diabetes protein antigen, a type I diabetes protein antigenic epitope polypeptide, and an immunosuppressor; the type I diabetes protein antigen is at least one of insulin, glutamic acid decarboxylase, and islet amyloid polypeptide, and the immunosuppressor is at least one of dexamethasone, cyclosporine A, tacrolimus, mycophenolate mofetil, azathioprine, prednisone, early prednisolone, anti-CD4 monoclonal antibody, and anti-CD3 monoclonal antibody.

### SCOPE

The present invention relates to composition for treating and/or preventing Type I diabetes and application thereof.

### BACKGROUND

Expert of World health Organization (WHO) predicted that diabetes would be the major threat to health in 21 century, even more dangerous threat than avian influenza and AIDS especially in Asia. WHO estimated that the diabetes patient will increase to around 200 million in 2010 and the number will be more than 330 million in 2025. Under the analysis of current situation, in next 10 years, 60% of patients will appear in Asia. As reported, world widely, most diabetes patients are in West Pacific area including China and East South of Asia which also including India. For the 5 countries, which have tremendous number of diabetes patient, there are 4 countries located in Asia.

During the development to wealth in China, the new incidence of diabetes increased years by years under the influence of nutrient like lipid, sugar taking and increasing number of ageing. In most area of China, the average increased incidence reached to 1/1000 compared with 90's of 20 century. The expert from China estimated that this increasing would be worse than what WHO anticipated In the future 10 years. According to the investigation in China, there are more than 25 million diabetes patients and 35 million patients with abnormal glucose tolerance. That means more than 60 million people with age over 20 will be affect by diabetes, in which the incidence of diabetes in rich areas and cities is higher than poorer areas and countryside, the overweight higher than the people with regular weight and the elders higher than young people. Also, new diabetes case appeared among people at age around 40, in which, the incident in those patients with age of more than 40 is about 87% of total diabetes patients and the peak age is around 50-70. Regarding the current age tendency, there exists variety markets with tremendous potential for diabetes drug developments.

Type I diabetes is a kind of autoimmune disease due to the malfunction of islet, which is characterized by invading of CD4⁺, CD8⁺T cells and macrophages into islet thus the insulin producing beta cells were destroyed by those invading. About 5-10% of diabetes account for TID (ADA [American Diabetes Association]. 1997. Report of the expert committee on the diagnosis and classification of diabetes mellitus. Diabetes Care 20:1183-1197; Atkinson M A, Leiter E H. 1999. The NOD mouse model of type I diabetes: As good as it gets? Nature 5:60601-604). The main mechanism of the TID is characterized by destroyed insulin producing cells by the auto-reactive T cell, which characterized by the CD4⁺, CD8⁺T cells and macrophages invading into the islet (Atkinson M A, Maclaren N K. 1994. The pathogenesis of insulin-dependent diabetes mellitus. N Engl J Med 331:1428-1436; Benoist C, Mathis D. 1997. Autoimmune diabetes: Retrovirus as trigger, precipitator or marker? Nature 388:833-834; Bjork S. 2001. The cost of diabetes and diabetes care. Diabetes Res Clin Pract 54(Suppl 1):13-18).

The inflammation was found in islet tissue of TID patient. After the lymphocyte invaded into islet of TID patient, the ICA, auto-reactive T cell against insulin, carboxy-peptidase and HSP were found in those patients.

The interaction of insulin B chain (aa 9-23) with MHC-11 that named as I-Ag7 was experimentally demonstrated. The TID or insulin dependent diabetes is a kind T cell mediated disease and as the result of destroyed islet with high glucose in blood due to self reactive cells. The B chain of insulin may be the auto-antigen candidate responsible for disease (Devendra, D. et al. Diabetes 54, 2549, 2005; Starwalt, S. et al. Protein Eng. 16, 147, 2003; Lee, L. et al. PNAS 102, 15995, 2005). The further experiment demonstrated that peptide 15-23 of insulin B chain could be recognized by T cells, and can be detected for production of interferon β and IL-17 in CD4 or CD8 T cell among TID patients. Other evidence shown that this peptide could react with CTL specific against to insulin B chain but not CD8 T cells in spleen (Hu, C. et al. J. Clin. Invest. 117, 3857, 2007; Amrani, A. et al. Nature 406, 739, 2000). Experiment demonstrated that the insulin C chain is another autoimmune antigen (Arif, T. I. Tree, T. R Astill et al. Autoreactive T cell responses show proinflammatory polarization in diabetes but a regulatory phenotype in health. The Journal of Clinical Investigation, vol. 113, no. 3, pp. 451-463, 2004).

Till to 1990, Beakkeskov demonstrated the 64 K antibody existed in TID patients' serum is auto-reactive antibody and T cells, thus the GAD is considered as the key antigen responsible for TID (Immune modulation for prevention of type I diabetes mellitus. Itamar Raz, Roy Eldor and Yaakov Naparstek. TRENDS in Biotechnology 23:128, 2005; Enee, E. et al. J Immunol 180, 5430, 2008; Xiurong Long, Wenbing Du, Zhongpu Su, Qinzheng Wei. Detection of glutamic acid decarboxylase antibodies in children with diabetes mellitus. Chinese Journal of Pediatrics. 1998, Vol 10.).

On the other hand, lots of reports demonstrated that the protein aggregations induced by islet amyloid polypeptide (IAPP) could destroy the membrane structure of islet beta cell, could induce apoptosis, could cause dysfunctions of islet beta cells and activate immune attacking of islet beta cells. And this protein aggregation was considered as the one of main cause for diabetes. The latest results showed that inhibiting the formation of IAPP could decrease the apoptosis of islet beta cell and increase the possibility of islet transplantation. As the consequence, the IAPP become the other promising target for TID drug development.

Currently, the treatment of TID mainly relies on insulin up-taking in which the patient needs daily injections of insulin. This treatment method is inconvenient for patients and may cause allergy and infections around the injection site. Also, insulin injection method can only partially lower the symptom of high glucose but cannot restore pancreatic function and prevents the pancreatic attack by auto-reactive T cells, thus can not control the long term neopathy. Considering the complication of diabetes, only 35-90% could be decreased by insulin injection approach. So the diabetes patients suffer those painful inconvenient and side effects. Therefore, it is necessary to develop the new approach to restore or improve the pancreatic function by inhibiting the autoirnmune responses towards the islet cells and let to potential cure in those TID patients especially for those young patient to improve theft life quality.

Since TID is an autoimmune disease caused by auto-reactive T cells, the immuno-suppressive agents were broadly used in clinic such as Dexamethasone (Dex), tacrolimus (FK506), cyclosporine (CsA), Mycophenolate mofetil (MMF), azathiopurine (Aza), prednisone (Pred), Methylprednisolone (MP), etc, and antibodies against anti lymphocyte globulin (ALG) and anti CD4 monoclonal antibody (OKT4). Those treatment approaches are nonspecific and may cause serious side effects to the patients. On the other hand, these treatments are very expensive in which the patients will spend several billion dollars each year. Also, those agents are non-specific immuno-suppressive agents and have toxicity or side effect if large dose were given. On the other hand, it may cause multiple neopathy and malfunction of organs due to the over inhibition of immune response. So it is urgent to develop target-specific drugs without toxicity or with minimum side effects.

### DISCLOSED INVENTION

The purpose of this invention is to provide the composition.

This invention provides a method and the active composition are list as 1) or 2) or 3):

1) Mixture of TID protein antigen and immune suppressive agents.

2) Disclosed mixture of TID epitope peptides and immune suppressive agents.

3) Disclosed mixture of TID protein antigen, epitope peptides and immune suppressive agents.

The active component of TID protein antigen, epitope peptides and immune suppressive agents can be packaged separately or packaged as one formulation.

At least one TID active component listed as Insulin, GAD and IAPP described as the protein antigen. Disclosed immune suppressive agents including but not limited to Dex, FK506, CsA, MMF, Aza, Pred, MP, anti CD4 monoclonal antibody and anti CD3 monoclonal antibody. At least one of those agents described as the immune suppressive agents for TID. Epitope peptides refer to single epitope peptide and multi epitopes polypeptide for TID.

In which, listed insulin may come from biological isolation of human, dog, cat, and genetic engineering recombinant protein. The insulin from human can be used for treating TID of cat and dog since insulin gene between human, cat, dog and mouse are very similar, in which, the homology of human and mouse is 95%, human and cat is 84%, human and cat is 89%.

The listed GAT may come from human, cat, dog and genetic engineering recombinant protein. The human GAT can be used for treating mouse TID and the gene homology between human and mouse is 90%.

The listed IAPP may come from human, cat, dog and genetic engineering recombinant protein.

The listed epitope peptides can be from human, cat, dog and can be chemically synthesized.

The listed protein antigen for TID can be specific to rh-insulin. The listed epitope peptides from rh-insulin refer to sequence 1 (this peptide named as B9-23), or sequence 2 (this peptide named as B15-23), or sequence 3 (this peptide named as C peptide), or sequence 12 (this peptide named as B23-29), or sequence 13 (this peptide named as B10-05).

The listed protein antigen for TID can be specific to dog insulin. The listed epitope peptides from dog insulin refer to sequence 4 (this peptide named as B9-23), or sequence 5 (this peptide named as B15-23), or sequence 12 (this peptide named as B23-29), or sequence 13 (this peptide named as B10-05).

The listed protein antigen for TID can be specific to cat insulin. The listed epitope peptides from cat insulin refer to sequence 6 (this peptide named as B9-23), or sequence 7 (this peptide named as B15-23), or sequence 12 (this peptide named as B23-29), or sequence 13 (this peptide named as B10-05).

The listed protein antigen for TID can be specific to human GAD65. The listed epitope peptide from human GAD65 refer to sequence 8 (this peptide named as G114-123).

The listed protein antigen for TID can be specific to human IAPP. The listed epitope peptide from human IAPP refer to sequence 9 (this peptide named as 1-36).

The listed protein antigen for TID can be specific to dog IAPP. The listed epitope peptide from dog IAPP refer to sequence 10 (this peptide named as 1-36).

The listed protein antigen for TID can be specific to cat IAPP. The listed epitope peptide from cat IAPP refer to sequence 11 (this peptide named as 1-36).

In which, sequence 1 is composed by 15 amino acids; sequence 2 is composed by 9 amino acids; sequence 3 is composed by 31 amino acids; sequence 4 is composed by 15 amino acids; sequence 5 is composed by 9 amino acids; sequence 6 is composed by 15 amino acids; sequence 7 is composed by 9 amino acids; sequence 8 is composed by 10 amino acids; sequence 9 is composed by 37 amino acids; sequence 10 is composed by 37 amino acids; sequence 11 is composed by 37 amino acids; sequence 12 is composed by 17 amino acids; sequence 13 is composed by 37 amino acids;

The composition of protein antigen and immune suppressive agents listed in 1) can be made as following ratios (quantity ratio) such as 1:20 to 20:1, 1:1 to 10:1. The specific ratios are 1:1 (10 μg protein antigen+10 μg immune suppressive agent) or 10:1 (10 μg protein antigen+1 μg immune suppressive agent) The composition of epitope peptide antigen and immune suppressive agents listed in 2) can be made as 1 g:1 g.

At least one function of provided composition in this invention listed below:

(1) Treatment or prevention of TID for vertebrate;

(2) Enhance the proliferation of CD4⁺CD25⁺Treg population in mammalian;

(3) Increase the ratio of CD4⁺CD25⁺Treg to CD4⁺T cells in mammalian;

(4) Increase the IL-10 secretion from T cells in mammalian;

(5) Inhibit the cytotoxicity effect of auto-reactive CD8⁺T cells in mammalian; The described inhibiting the cytotoxicity effect of CD8⁺T cells refer to cytotoxicity against islet cells or/and spleen cells.

(6) Control the blood glucose for TID individuals (mammalian);

(7) Up-regulate the transcription level of IL-10 or/and TGF-β in PBMC or/and spleen;

(8) Inhibit the DC maturation; The described inhibiting the DC maturation refer to down-regulating at least one of surface proteins (CD40, CD80, CD83, CD86) in DC;

The listed vertebrates are mammalian that can be specific to mouse, cat, dog or human.

The application of products of this composition and methods for treatment or/and prevention of TID listed in invention, also is protected by this invention

The protection area in this invention is listed in below a)-h) product areas when the composition and methods described in this invention are applied into treatment or/and prevention products.

a) Increase the ratio of CD4⁺CD25⁺Treg and CD4⁺T cells in mammalian;

b) Enhance the proliferation of CD4⁺CD25⁺Treg population in mammalian;

c) Increase the IL-10 secretion from T cells in mammalian;

d) Control the blood glucose for TID in mammalian;

e) Inhibit the cytotoxicity effect of auto-reactive CD8⁺T cells;

f) Up-regulate the transcription level of IL-10 or/and TGF-β in PBMC or/and spleen;

g) Inhibit the DC maturation in mammalian;

h) Decrease the secretion of at least one of surface proteins (CD40, CD80, CD83, CD86) in DC;

The listed vertebrates are mammalian that can be specific to mouse, cat, dog or human.

The application of pharmaceutical composition for treatment or/and prevention of TID listed in invention, also is protected by this invention

The pharmaceutical composition in invention can inhibit the TID by inducing CD4⁺ CD25⁺ Treg after administration of composition listed in invention.

The pharmaceutical composition listed in invention can be made as following ratios when applied, 1) Protein antigen for TID, the concentration of insulin will be 0.01˜1 IU per Kg of body weight, eg. 0.15˜0.25 IU per Kg body weight, the concentration of immune suppressive agent Dex will be 0.01˜600 μg per Kg body weight, eg. 1˜511 g per Kg body weight;

2) As the TID epitope peptide antigen, the concentration of B23-29 will be 0.05˜1 μg per Kg of body weight, eg. 0.05˜1 μg per Kg body weight, the concentration of immune suppressive agent Dex will be 0.01˜1 μg per Kg body weight, eg. 0.05˜0.2 μg per Kg body weight;

3) As the TID epitope peptide antigen, the concentration of B10-05 will be 0.05˜1 μg per Kg of body weight, eg. 0.05˜1 μg per Kg body weight, the concentration of immune suppressive agent Dex will be 0.01˜1 μg per Kg body weight, eg. 0.05˜0.2 μg per Kg body weight;

4) As the TID protein antigen the concentration of GAD will be 0.05˜1 μg per Kg of body weight, eg. 0.05˜1 μg per Kg body weight, the concentration of immune suppressive agent Dex will be 0.01˜1 μg per Kg body weight, eg. 0.05˜0.2 μg per Kg body weight;

5) As the TID epitope peptide antigen, the concentration of G114-123 will be 0.05˜1 μg per Kg of body weight, eg. 0.05˜1 μg per Kg body weight, the concentration of immune suppressive agent Dex will be 0.01˜1 μg per Kg body weight, eg. 0.05˜0.2 μg per Kg body weight;

The composition and methods for treatment or/and prevention of TID in this invention can be delivered by injection, ejection, instillation, infiltration, absorption or physical and chemically delivered by im, id, sc, iv, intra mucosal; or delivered by combining with other substrate mixed or packaged.

The composition can be administrated every 3-30 days and total 4-8 administrations.

### FIGURE LEGENDS

FIG. 1. The dose effects of composition and methods on Treg population. The NOD mice were immunized with different doses of genetic engineered recombinant human insulin (rh-insulin) and DEX, the percentage of CD4⁺CD25⁺Treg was measured in spleen. A. FACS result; B. Statistical analysis of FACs data. In Fig A and B, 1 refer to group1 (10+10) in which 10 μg rh-insulin and 10 μg DEX mixed treatment; 2 refer to group 2 (100+100) in which 100 μg rh-insulin and 100 μg DEX mixed treatment; 3 refer to group 3 (500+100) in which 500 μg rh-insulin and 100 μg DEX mixed treatment; 4 refer to group 4 in which the NOD mice without treatment.

FIG. 2. The dose effect of composition and methods on Treg proliferation. The NOD mice were immunized with different doses of rh-insulin and DEX. The purified CD4⁺CD25⁺Treg from spleen were labeled with CFSE and stimulated with rh-insulin or human insulin epitope peptide hB9-23 in vitro. After stimulation, the proliferation of Treg was measured. A. FACS result; B. Statistical analysis of FACs data. In Fig A and B, 1 refer to positive control in which the cell treated with anti-CD3 antibody; 2 refer to non-related control in which the cell treated with OVA323-339; 3 refer to negative control (The cells tested in 1, 2, 3 are from unimmunized NOD mice); 4 refer to stimulated group in which the cell stimulated with 10 μg rh-insulin and 10 μg DEX mixture; 5 refer to stimulated group in which the cell stimulated with 100 μg rh-insulin and 100 μg DEX mixture; 6 refer to stimulated group in which the cell stimulated with 500 μg rh-insulin and 100 μg DEX mixture; 7 refer to stimulated group in which the cell stimulated with 10 μg human insulin epitope peptide B9-23 and 10 μg DEX mixture; 8 refer to stimulated group in which the cell stimulated 100 μg human insulin epitope peptide B9-23 and 100 μg DEX mixture; 9 refer to stimulated group in which the cell stimulated 500 μg human insulin epitope peptide B9-23 and 100 μg DEX mixture;

FIG. 3. The dose effects of composition and methods on expression of IL-10. The NOD mice were immunized with different doses of rh-insulin and DEX. The purified CD4⁺CD25⁺Treg from spleen was stimulated with rh-insulin or human insulin epitope peptide hB9-23 in vitro. After stimulation, the level of IL-10 from supernatant was measured (pg/ml). 1 refer to positive control in which the cell treated with anti-CD3 antibody; 2 refer to non-related control in which the cell treated with OVA323-339; 3 refer to negative control (The cells tested in 1, 2, 3 are from unimmunized NOD mice); 4 refer to stimulated group in which the cell stimulated with 10 μg rh-insulin and 10 μg DEX mixture; 5 refer to stimulated group in which the cell stimulated with 100 μg rh-insulin and 100 μg DEX mixture; 6 refer to stimulated group in which the cell stimulated with 500 μg rh-insulin and 100 DEX mixture; 7 refer to stimulated group in which the cell stimulated with 10 μg human insulin epitope peptide B9-23 and 10 μg DEX mixture; 8 refer to stimulated group in which the cell stimulated 100 μg human insulin epitope peptide B9-23 and 100 μg DEX mixture; 9 refer to stimulated group in which the cell stimulated 500 μg human insulin epitope peptide B9-23 and 100 μg DEX mixture;

FIG. 4. The blood glucose level in acute TID after treatments. The change of blood glucose (mM) was measured in NOD mice induced by STZ with clinic sign of TID after treated with high dose (100 μg rh-insulin and 100 μg DEX) and low dose (10 μg rh-insulin and 10 μg DEX). Empty circle with solid line refer to the TID group without treatment; Filled circle with solid line refer to the TID group with low dose treatment (10+10); Empty circle with dash line refer to the TID group with high dose treatment (100+100);

FIG. 5. The CTL response in acute TID after treatments. The CTL response was measured in NOD mice induced by STZ with clinic sign of TID after treated with high dose (100 μg rh-insulin and 100 μg DEX) and low dose (10 μg rh-insulin and 10 μg DEX). The CFSE labeled spleen cells were incubated with CD8 T cell epitope peptide of insulin 10-18 and were transferred into the NOD mice with treatment. The CTL response was measured 12 hour later of transfer. A. FACS result; B. Statistical analysis of FACs data. In Fig A and B, 1 refer to group1 CTL result in which the NOD mice show clinical sign; 2 refer to group 2 (10+10) in which 10 μg rh-insulin and 10 μg DEX mixed treatment; 3 refer to group 3 (100+100) in which 100 μg rh-insulin and 100 μg DEX mixed treatment; 4 refer to group 3 (100+100) in which the NOD mice pre-injected with anti-CD8 antibody were immunized with 100 μg rh-insulin and 100 μg DEX.

FIG. 6. The CTL response relationship between levels of blood glucose and auto-reactive CTL in acute TID after treatment. In which each dot refer to single mouse. CTL response in TID mice after treatment listed composition.

FIG. 7. The level of Treg (Foxp3⁺CD4⁺) in acute TID after treatments. The ratio of Treg (Foxp3⁺CD4⁺) with CD4+ T cells was measured in NOD mice induced by STZ with clinic sign of TID after treated with high dose (100 μg rh-insulin and 100 μg DEX) and low dose (10 μg rh-insulin and 10 μg DEX). 1 refer to control group in which the NOD mice show no clinical sign and no treatment; 2 refer to group 1 in which the NOD mice show clinical sign; 3 refer to group 2 (10+10) in which 10 μg rh-insulin and 10 μg DEX mixed treatment; 4 refer to group 3 (100+100) in which 100 μg rh-insulin and 100 μg DEX mixed treatment.

FIG. 8. The survival curve of long term TID after treatments. When the NOD mice show clinic sign of TID, the survive rate was recorded timely after NOD mice with TID were treated with low dose (10 μg rh-insulin and 10 μg DEX). Empty circle with solid line refer to group 1 (Diabetic group without treatment); Filled circle with solid line refer to group 2 (Treatment group).

FIG. 9. The level of blood glucose change result of long term TID after treatments. After NOD mice were induced by STZ with clinic sign of TID, the level of blood glucose was measured after the mice were treated with low dose (10 μg rh-insulin and 10 μg DEX). Empty circle with solid line refer to group 1 (Diabetic group without treatment); Filled circle with solid line refer to group 2 (Treatment group);

FIG. 10. The CTL response against insulin10-18 in long term TID. After NOD mice were induced by STZ with clinic sign of TID, the mice were treated with low dose (10 μg rh-insulin and 10 μg DEX). The CFSE labeled cells were incubated with CD8 T cell epitope peptide of insulin10-18 and were transferred into the NOD mice with treatment. The CTL response was measured 12 hour later of transfer. 1 refer to CTL result of group 1(Diabetic group without treatment); 2 refer to CTL result of group 2 (Treatment group) in which 10 μg rh-insulin and 10 μg DEX mixed treatment; 3 refer to group 3 in which the CTL were blocked with anti-CD8 mAb.

FIG. 11. The CTL response against islet cells in long term TID. After NOD mice were induced by STZ with clinic sign of TID, the mice were treated with low dose (10 μg rh-insulin and 10 μg DEX). The CFSE labeled islet cells were transferred into the NOD mice with treatment. The CTL response was measured 12 hour later of transfer. 1 refer to CTL result of group 1(Diabetic group without treatment); 2 refer to CTL result of group 2 (Treatment group) in which 10 μg rh-insulin and 10 μg DEX mixed treatment;

FIG. 12. The dose effects of the treatment on expressions of IL-10 and TGF-β in rabbits. After the rabbits were treated with different doses of rh-insulin and DEX. The spleens were isolated and splenocytes were stimulated with rh-insulin in vitro. After stimulation, the level of IL-10 and TGF-β were measured by RT-PCR. A, result of RT-PCR electrophoresis from PBMC, lane 1 refer to group 1 (100+100) in which 100 μg rh-insulin and 100 μg DEX mixed treatment; lane 2 refer to group 2 (10+10) in which 10 μg rh-insulin and 10 μg DEX mixed treatment; lane 3 refer to group 3 (10+1) in which 10 μg rh-insulin and 1 μg DEX mixed treatment; lane 4 refer to group 4 (DEX100) in which 100 μg DEX treatment; lane 5 refer to group 5 (Ins100) in which 100 μg rh-insulin treatment; lane 6 refer to group 6 (Negative control) in which 100 ul PBS treatment. B, result of RT-PCR from PBMC and spleen, B1-1 refer to RT-PCR of IL-10 from PBMC; B1-2 refer to RT-PCR of TGF-β from PBMC; B2-1 refer to RT-PCR of IL-10 from spleen; B2-2 refer to RT-PCR of TGF-β from spleen; In B1-1, B1-2, B2-1 and B2-2, lane 1 refer to group 1 (100+100) in which 100 μg rh-insulin and 100 μg DEX mixed treatment; lane 2 refer to group 2 (10+10) in which 10 μg rh-insulin and 10 μg DEX mixed treatment; lane 3 refer to group 3 (10+1) in which 10 μg rh-insulin and 1 μg DEX mixed treatment; lane 4 refer to group 4 (DEX100) in which 100 μg DEX treatment; lane 5 refer to group 5 (Ins100) in which 100 μg rh-insulin treatment.

FIG. 13. The dose effect of treatment on expressions of IL-10 and TGF-β in dogs. After TID dog were treated with different doses of rh-insulin and DEX. Their splenocytes was isolated at days −3, 0, 8, 20, 28 and stimulated with rh-insulin in vitro. The level of IL-10 and TGF-β were measured by RT-PCR. A, Result of IL-10 levels, B, Result of TGF-β levels. The 1, 2, 3, 4, 5 in A and B stand for RT-PCR result of sample on days −3, 0, 8, 20, 28.

FIG. 14. The effect of the treatment on Treg cell population in acute TID dogs. The Alloxan induced TID dogs were treated with rh-insulin and DEX, the percentage of CD4⁺ CD25⁺Treg among all cells in pancreas was measured. A, Ratio of Treg to CD4+ T cells; B, Ratio of Treg to all cells; In A and B, 1, refer to diabetic animals; 2, refer to group treated with 100 μg rh-insulin and 1.5 μg DEX.

FIG. 15. The survival of TID dogs after the treatments. After TID dogs were treated with rh-insulin and DEX or rh-insulin and CsA, their survival was followed. 1 refer to dogs treated with rh-insulin at 0.15 IU/kg body weight and DEX at 1 ug/Kg body weight; 2 refer to dogs treated with rh-insulin at 0.15 IU/kg body weight and CsA at 100 ug/Kg body weight; 3 refer to diabetic model control. The grey line indicates treatment duration, in which 3 injections applied to each cycle of treatment.

FIG. 16. TID dog blood glucose level change after the treatments. After TID dogs were treated with rh-insulin and DEX, the level of blood glucose was followed. Solid line refers to dogs treated with rh-insulin at 0.15 IU/kg body weight and DEX at 1 ug/Kg body weight; Dashed line refers to diabetic model control. The grey line indicates treatment duration, in which 3 injections applied to each cycle of treatment. Vertical line is for defining the high level of blood glucose.

FIG. 17. TID dog blood glucose level change after the treatments. After TID dogs were treated with rh-insulin and CsA, the level of blood glucose was followed. Solid line refers to dogs treated with rh-insulin at 0.15 IU/kg body weight and CsA at 100 ug/Kg body weight; Dash line refers to diabetic model control. The grey line indicates treatment duration, in which 3 injections applied to each cycle of treatment. Vertical line is for defining the high level of blood glucose.

FIG. 18. TID dog body weight loss after the treatments. After TID dogs were treated with rh-insulin and DEX, their body weight changes were followed. 1 refer to dogs treated with rh-insulin at 0.15 IU/kg body weight and Dex at 1 ug/Kg body weight; 2 refer to dogs treated with rh-insulin at 0.15 IU/kg body weight and CsA at 100 ug/Kg body weight; 3 refer to diabetic model control. The grey line indicates treatment duration, in which 3 injections applied to each cycle of treatment.

FIG. 19. The expression of CD40 and IL-10 in DC converted from PBMC. The isolated PBMC from normal people and TID patients were induced into CD1a⁺ DC by GM-CSF and IL-4. After CD1a⁺ DCs were stimulated with insulin and DEX, the expression of CD40 and IL-10 were measured. In which, A refer to the result of CD40 expression; B refer to the result of IL-10 expression. The 1-1 and 1-2 in A and B refer to result of 2 TID patient; the 2-1, 2-2 and 2-3 in A and B refer to result of 3 normal human samples; In 1-1, 1-2, 2-1, 2-2 and 2-3, 1, refer to negative control; 2 refer to samples treated with rh-insulin at final concentration of 10 μg/ml; 3 refer to samples treated with DEX at final concentration of 10 μg/ml; 4 refer to samples treated with rh-insulin and DEX at final concentration of 10 μg/ml.

FIG. 20. The high throughput screening results from massive samples for the pharmaceutical composition. The expression profile of CD40, CD80, CD83 and CD86 on DC after human PBMC treated with composition. 1 refer to negative control; 2 refer to samples treated with rh-insulin at final concentration of 10 μg/ml; 3 refer to samples treated with both rh-insulin and DEX at final concentration of 10 μg/ml; 4 refer to samples treated with both rh-insulin and Rap at final concentration of 10 μg/ml; 5 refer to samples treated with both rh-insulin and CsA at final concentration of 10 μg/ml; 6 refer to samples treated with both rh-insulin and FK506 at final concentration of 10 μg/ml; Each dot stands for single blood sample.

### EXAMPLE OF EXPERIMENTS

The regular procedure applied if no specific statements addressed in following examples.

All materials and reagents in following procedure are commercially available if no specific statement addressed.

Sources of composition for the treatments: The rh-insulin was from Novo Nordisk A/S; 1 IU rh-insulin equal to 45.4 μg in quantity. The peptides of insulin were synthesized by Beijing Aoke in which the B9-23 peptide from sequence 1 refer to epitope peptide for TID; All immune-suppressors were GMP grade and produced by China Pharma Group. In which DEX is H34023626, Rap is 5R039501, FK506 is H20080457 and CsA is H10940045. Composition was made by protein, or peptide mixed with immune-suppressors before injection.

### Example 1

### Dose Effect of Composition and Methods on NOD Mice

Immunizations of NOD Mice

1, Immunization with Pharmaceutical Composition of Rh-Insulin and DEX.

The NOD mice were divided into 4 groups with 3 mice per group. The pharmaceutical composition of rh-insulin and DEX were administrated into each group from s.c on days 1, 4 and 7. Group 1(10+10) was injected with 10 μg of rh-insulin and 10 μg of DEX dissolved in 100 ul of PBS. Group 2 (100+100) was injected with 100 μg of rh-insulin and 100 μg of DEX dissolved in 100 ul of PBS. Group 3 (500+100) was injected with 500 μg of rh-insulin and 100 μg of DEX dissolved in 100 ul of PBS. All animals were injected on days 1, 4 and 7.

2 Immunization with Pharmaceutical Composition of Insulin Epitope Peptides B9-23 and DEX.

The NOD mice were divided into 4 groups with 3 mice per group. The pharmaceutical composition were delivered into each group from s.c on day 1, 4, 7. Group 1 (10+10) was injected with 10 μg of human insulin epitope peptides B9-23 and 10 μg of DEX dissolved in 100 ul of PBS. Group 2 (100+100) was injected with 100 μg of human insulin epitope peptides B9-23 and 100 μg of DEX dissolved in 100 ul of PBS. Group 3 (500+100) was injected with 500 μg of human insulin epitope peptides hB9-23 and 100 μg of DEX dissolved in 100 ul of PBS. All animals were injected days 1, 4 and 7.

Determination of Dose Effect of Pharmaceutical Composition by Measuring the Population and Proliferation of Treg and IL-10 Expression.

1. The Dose Effect of Pharmaceutical Composition on Treg Population.

The drug effect of suppressive was determined with percentage of Treg population in immunized NOD mice at day 8 after the last immunization.

Detail procedure listed below:
1) The spleen was taken out and placed into dish containing 2 ml of RPMI-1640 under standard sterilization protocol.
2) The spleen was placed on sterilized bronze container and smashed by front end of syringe.
3) The single suspension was filtered into 15 ml tube and spin down at 2000 rpm for 10 minutes.
4) The supernatant was discarded, 2-3 ml red blood cell lysis buffer added into the cell pellet and react for 2 min before stopped by adding equal volume of RPMI-1640. Spin down at 2000 rpm for 10 minutes.
5) The supernatant was discarded, 3-4 ml of RPMI-1640 with 2% of FBS was added and the cell pellet was re-suspended.
6) The cell solution was filtered by glass fiber column to remove the B cells.
7) Counted the cell density.
8) Wash the cell once with PBS and adjust the cell density at 2×10⁷
9) 10⁶ cells were stained with CD4 and CD25 mAb by adding 0.2111 of PE-anti-CD4 mAb and 0.2111 of APC-anti-CD25 (eBioscience 12-0041, 17-0251) at RT for 10 minutes along with light avoiding. The percentage of Treg was measured by FACs compared with cells from NOD mice without immunization.

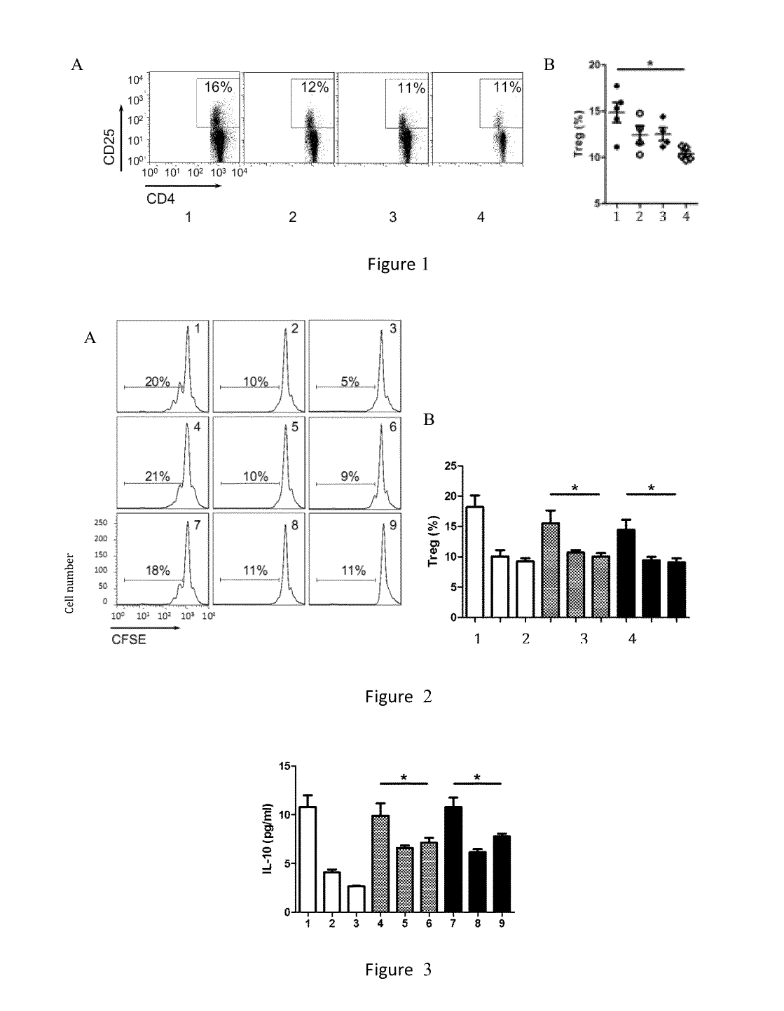

The result in FIG. 1 shows the group 1 (10+10) injected with 10 μg of rh-insulin and 10 μg of DEX increase Treg frequency up to 16%, whereas only 10-12% of Treg maintained in other groups.

2. The Dose Effect of Pharmaceutical Composition by Detecting Treg Proliferation.

The pharmaceutical composition effect was determined with proliferation of Treg against insulin (rh-insulin and DEX) and B9-23 (human insulin epitope peptide B9-23 and DEX) in immunized NOD mice at day 8 after the last immunization. In which, the T cells were stained with CFSE and the proliferation of Treg was measured by FACs.

Detail procedure listed below:

Steps 1-9 are same as method mentioned above whereas 10) the spleen cells were stained with 3 μM CFSE at room temperature (RT) for 8 minutes with shaking in step 100
11) Adding equal volume of FBS to stop the staining reaction. Spin down and wash the cells for 3 times.
12) Add the cells into 96-well plate and each well add 2×10⁵ cells. Then 100 μl Anti-CD3 mAb (AbDSerotec, MCA500GA) with final concentration at 1 μg/ml were added into one well as positive control, one well was added with 5 μg/ml of OVA323-339 (ISQAVHAAHAEINEAGR) as the un-related control. One well was added with 10 μg/ml of human insulin epitope peptides hB9-23 to stimulate cell proliferate. Also the cells without CFSE staining were added into the well as the control.
13) Incubate the cells at 37° C., 5% CO₂ for 3 days. FACs measured the proliferation of Treg in comparison with untreated and non-diseased NOD mice.

The result in FIG. 2 shows that group 1 (10+10) injected with 10 μg of rh-insulin plus 10 μg of DEX or 10 μg of human insulin epitope peptides hB9-23 plus 10 μg of DEX significantly enhanced Treg proliferation comparing with other groups.

3. Dose Effect of Pharmaceutical Composition by Detecting IL-10 Expression in Treg.

The pharmaceutical composition effect was determined by IL-10 expression of Treg in NOD mice immunized with 10 μg of rh-insulin plus 10 μg of DEX and 10 μg of human insulin epitope peptides B9-23 plus 10 μg of DEX at day 8 after the last immunization.

Detail procedure listed below:

Steps 1-9 are Same as Method Mentioned Above.
10) Add the cells into 96-well plate and each well add 2×10⁵ cells. Then 1000 Anti-CD3 mAb with final concentration at 1 μg/ml were added into one well as positive control, one well was added with 5 μg/ml of OVA323-339 as the un-related control. One well was added with 10 μg/ml of human insulin epitope peptides hB9-23 to stimulate cell. One well was added with 10 μg/ml of rh-insulin to stimulate cell. Also the cells without stimulation were added into the well as the negative control.
11) Incubate the cells at 37° C., 5% CO₂ for 24 hours. The 30 ul supernatant were collected and mixed with 30 ul PBS which contained 0.10 FlexSet microbead (BD, 558300) for 30 minutes. The 30 ul PBS that contained 0.10 PE labeled antibody were added and incubated for another 30 minutes. FACs measured the IL-10 expression in comparison with nave NOD mice.

The result in FIG. 3 shows that group 1 (10+10) injected with 10 μg of rh-insulin plus 10 μg of DEX or 10 μg of human insulin epitope peptides hB9-23 plus 10 μg of DEX significantly increased the expression of IL-10.

In summary, the results from this example show that rh-insulin plus DEX, or human insulin epitope peptides plus DEX can induce Treg production in NOD mice. This Treg can be proliferated and can produce IL-10 caused by human insulin and human insulin epitope peptide B9-23. The best dose for this effect is 10 μg of rh-insulin plus 10 μg of DEX and 10 μg of human insulin epitope peptides B9-23 plus 10 μg of DEX

### Example 2

### Treatment Effect of Pharmaceutical Composition on Acute TID NOD Mice

NOD Mice Induction and Immunizations

After the dose of pharmaceutical compositions determined in example 1, the rh-insulin plus DEX were inject into diabetic NOD mice of which levels of blood glucose were more than 12 mmol by i.p.

For establishment of TID model, 18 NOD mice were induced by i.p. injections with 40 mg/kg STZ (Sigma Aldridge, 50130) for 5 continuous days. When the level of blood glucose reached higher than 12 mmol, those NOD mice were divided into 3 groups with 6 mice per group. The group 1 (diabetic group) was un-treatment group. Group 2 (10+10) was treated with 10 μg of rh-insulin plus 10 μg of DEX. Group 3 (100+100) was treated with 100 μg of rh-insulin plus 100 μg of DEX. The pharmaceutical compositions were injected from i.p. on days 1, 4 and 7 after the mice were determined as TID mice.

Detection of Lood Glucose Levels Change, CTL and Treg.

1. Detection of Blood Glucose Levels Change.

The blood glucose was monitored on days 5, 7, 11, 17, 19, 24, 28, 32 and 37 to reflect the pharmaceutical composition effect after injection

Test procedure: 10 ul of blood samples were dropped to blood glucose test strips and a test instrument read the concentration of blood glucose.

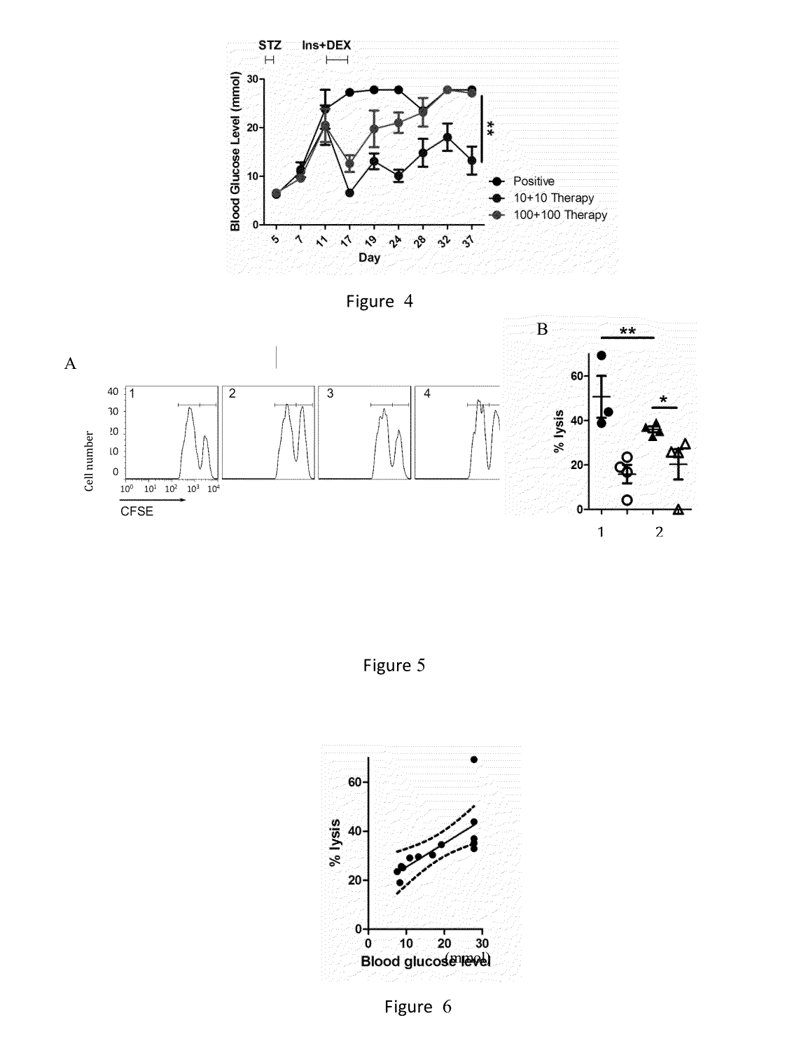

The result in FIG. 4 shows that group 2 (10+10) injected with 10 μg of rh-insulin plus 10 μg of DEX can control the level of blood glucose at 10-12 mmol whereas group 3 (100+100) injected with 100 μg of rh-insulin plus 100 μg of DEX show decreasing glucose level at the beginning of treatment, but increased at the same level as diabetic group finally.

2. Detection of CTL Response.

The induced Treg cells could suppress auto-reactive CD8 T cell responses after pharmaceutical composition injections. The CTL responses were detected on day 37.

Detail procedure listed below:
1) The CD8 T cells were depleted by injection of anti-CD8 mAb (eBioscience, clone 53-6.7) on day 35 and 36 for mice immunized with 100 μg of rh-insulin plus 100 μg of DEX.
2) The spleen cells from normal untreated NOD mice were isolated and counted, the methods are same as mentioned above 1)-9).
3) The equal number of cells was stained with 5 μM and 20 μM of CFSE for 8 minutes at RT. The staining stopped by adding equal volume of FBS followed by 3 time washes.
4) The cells stained with 20 μM of CFSE were incubated with 50 μg/ml of Insulin10-18 CD8 T epitope peptide (HLVEALYLV) at 37° C., 5% CO₂ for 30 min followed by wash.
5) Mix equal number of cells stained with 5 μM and 20 μM of CFSE and adopt transfer the mixed cell into NOD mice described in step 1.
6) The spleen cell were isolated after 12 hours' transfer, the specific lysis of target was measured by

FACs and formula (Specific lysis ratio=1-target cells/control cellsx100%) was applied.

The result in FIG. 5 shows that group 2 (10+10) injected with 10 μg of rh-insulin plus 10 μg of DEX can significantly suppress the CTL responses from auto-reactive CD8 T cells, whereas the CTL responses from the group injected with 100 μg of rh-insulin plus 100 μg of DEX showed less degree of the suppression. This CTL response can be blocked by CD8 antibody, which was apparently correlated with the level of blood glucose and thus demonstrated that the controlled blood glucose was due to the improved autoimmune activity.

3. Detection of Mouse Treg Population.

The Treg cells can be induced by pharmaceutical composition treatments, thus the Treg population was detected on day 37.

Detail procedures are same as part 2 of example 1.

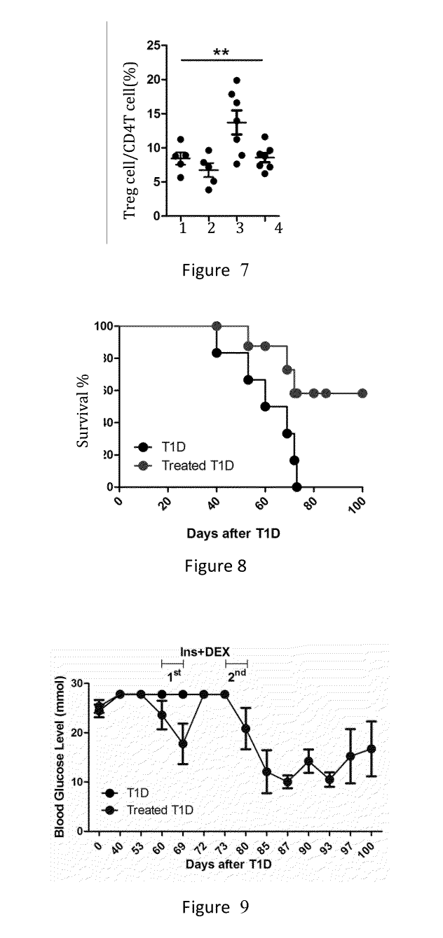

The result in FIG. 7 shows that group 2 (10+10) injected with 10 μg of rh-insulin plus 10 μg of DEX can increase Treg frequency to 15%, whereas only 8-10% of Treg in other groups.

In summary, the result from this example shows that rh-insulin plus DEX can induce Treg production in NOD mice and this Treg population can be explained that cease of CTL activities and islet attacking from the auto-reactive CD8 T cells by such induction of Treg cells.

### Example 3

### Effect of Pharmaceutical Composition on Long-Term TID NOD Mice

The example 2 demonstrated that the pharmaceutical composition possess the effect against short term TID. The following example shows the long-term effect of the pharmaceutical composition treatment.

NOD Mice Induction and Immunizations

For establishment of TID model, 16 NOD mice were induced by i.p. injection of 40 mg/kg STZ for 5 continuous days. After 2 month NOD mice showed the diabetic symptom, animals were divided into 2 groups with 8 mice per group. The group 1 (diabetic group) was un-treatment group. Group 2 (treatment group) was treated with 10 μg of rh-insulin plus 10 μg of DEX. The pharmaceutical composition was injected from i.p on days 1, 4 and 7 as one treatment cycle. A second treatment cycle was used one week later after the first cycle.

Detection of Survival, Blood Glucose Levels Change and CTL.

1. Survival of TID NOD Mice.

The survival was observed timely for 100 days after treatments of TID NOD mice.

The result in FIG. 8 shows that injections with rh-insulin plus DEX can make 60% of TID NOD mice survived on day 100, whereas all animals dead on days 60 to 80 in the untreated group.

2. Blood Glucose Levels Change Detection.

The levels of blood glucose were monitored on days 0, 40, 53, 60, 69, 72, 73, 80, 85, 87, 90, 93, 97 and 100 to reflect the pharmaceutical composition effect.

Test procedure: 10 ul of blood samples were spotted onto the test strips and the test instrument read the concentration of blood glucose.

The result in FIG. 9 shows that group 2 injections with rh-insulin plus DEX can control the level of blood glucose at 10-15 mmol, whereas no control in the control group 1.

3. Mice CTL Response Detection.

The CTL response was detected on day 60 for the control group and on day 100 for the treatment group.

Detail procedures are same as part 2 of example 2.

Additionally, the CTL response against islet cell was measured. In which the pancreatic cell isolated from nave NOD mice used as target cell. The specific procedures are: 1) The NOD mice were sacrificed and the pancreatic tissues and liver exposed under sterilized condition. 2) The 10 ml 1 mg/ml of Collagenase P (Roche, Cat. No. 11213857001) were injected into pancreatic tissue and digested for 1 hour at 37° C. 3) The pancreatic cells were washed twice and spin down at 250 g for 1 minute. 4) The cell pellets were re-suspended with 3 ml of 25% Ficoll (Roche) and in turns 2 ml of 23% Ficoll, 2 ml of 20% Ficoll, 2 ml of 11% Ficoll were added into above the 23% Ficoll. Then spin down the cell solution at 800 g for 10 minutes and the islet cell phase were taken out, the Ficoll solution in islet cell was washed out with twice PBS washing. 5) The islets were digested with 0.25% of trypsin for for 10 minutes at 37° C. 6) The single suspension islet cells were stained with 20 uM CFSE as the target cells whereas the effect cells stained with 5 uM CFSE were added into the target cells.

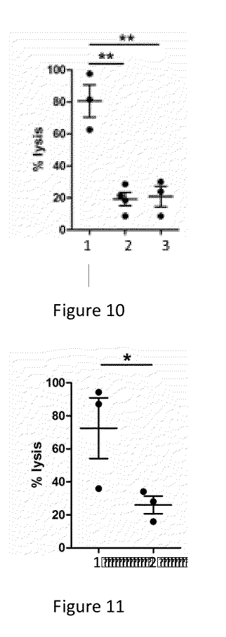

The result shows that the auto-reactive CD8 T cells against insulin 10-18 were significantly inhibited in the treated group, but not in the control group. The inhibition effect can be blocked by anti-CD8 mAb (FIG. 10). Similarly, the CTL response against islet cells was also controlled after the treatments (FIG. 11).

### Example 4

### Dose Effect of Pharmaceutical Composition in Rabbits

It demonstrated in example 1-3 that rh-insulin and DEX have therapeutic effect against TID in NOD mice, and then the effect was evaluated in rabbit.

Immunization of Rabbits

18 rabbits were divided into 6 groups with 3 rabbits per group. The pharmaceutical composition was injected i.p on days 1, 4 and 7 as one treatment cycle. The group 1 (100+100) was treated with 100 μg of rh-insulin plus 100 μg of DEX. Group 2 (10+10) was treated with 10 μg of rh-insulin plus 10 μg of DEX. Group 3 (10+1) was treated with 10 μg of rh-insulin plus 1 μg of DEX. Group 4 (DEX100) was treated with 100 μg of DEX alone. Group 5 (Ins100) was treated with 10 μg of rh-insulin alone. Group 6 (negative control) was treated with 100 ul of PBS. The second cycle was applied two week later (Day 21, 24, 27).

Detection of Suppressive Cytokine IL-10 and TGF-β

The expression level of IL-10 and TGF-β were measured from rabbit PBMCs and spleens on day 2 after last immunization (day 28).

Specific procedures are: 1) The PBMCs from immunized rabbits were isolated and purified by Ficoll, in which 4 ml of Ficoll400 (Sigma) were added under the 8 ml of rabbit blood and spin down at 1500 rpm for 15 minutes. The PBMC phase was taken out and washed after spin down. Then the PBMC were stimulated with 10 μg/ml of rh-insulin in vitro. 2) The spleen cells from immunized rabbit were collected and red blood cell removed by adding 2 ml lysis buffer (Biyuntian) into cells suspension for 2 minutes. Adding equal volume of FBS to stop the lysis reaction. The spleen cells were counted and stimulated with 10 μg/ml of rh-insulin. 3) 24 hours later of stimulation, the stimulated cells and PBMC were collected and Trizol added into the cell solution for RNA extraction. 4) The RNA were purified and transcribed into cDNA by Toyobo ReversTraAce kit according to the instruction. 5) The primers for HPRT, IL-10 and TGF-β amplification were designed as bellows:

HPRT p1: 5′-CCATCACATTGTAGCCCTCTGT-3′
HPRT p2: 5′-CTTGCGACCTTGACCATCTTT-3′
IL-10 p1: 5′-TATGTTGCCTGGTCTTCCTGG5-3′
IL-10 p2: 5′-CTCCACTGCCTTGCTCTTGT-3′
TGF-β p1: 5′-AACAAGAGCAGAAGGCGAATG-3′
TGF-β p2: 5′-ACAGCAAGGAGAAGCGGATG-3′

The endogenous gene encoding the HPRT as a reference gene was amplified and adjusted to the same level for each sample. 6) The IL-10 and TGF-β were amplified by PCR and the expressions were analyzed by DNA gel running and Gelpro software.

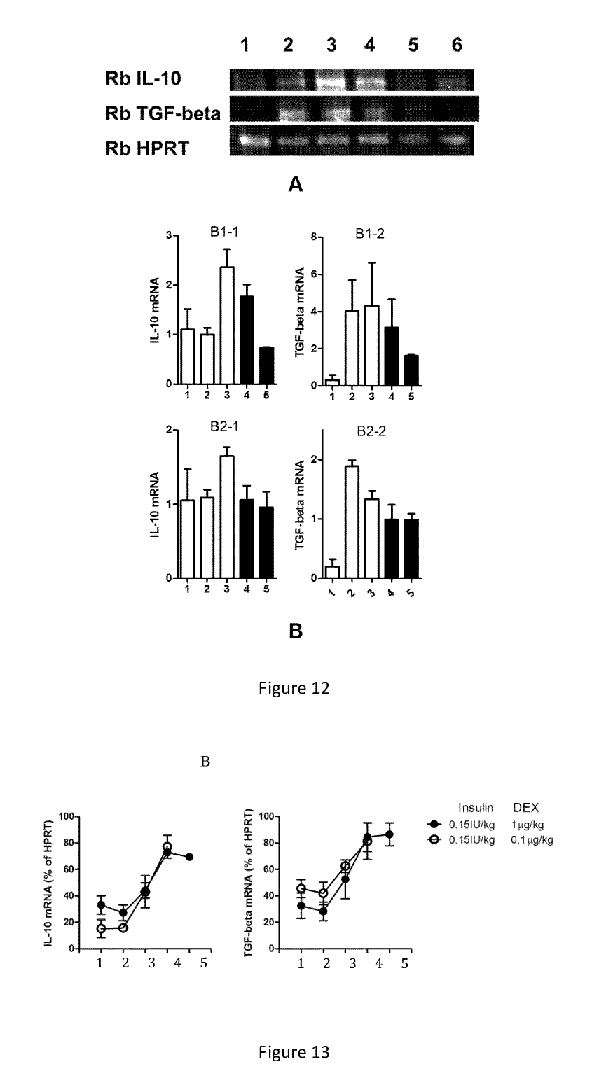

The result in FIG. 12 shows that group 3 (10+1) injected with 10 μg of rh-insulin plus 1 μg of DEX can induce highest and less high amount of IL-10 and TGF-β in spleen and PBMC in the treated animals.

### Example 5

### Dose Effect of Pharmaceutical Composition in Dogs

It demonstrated in example 4 that rh-insulin and DEX have therapeutic effect against TID in rabbit, then the effect was evaluated in dogs.

### Immunization of Dogs.

Dogs used to induce TID model were injected with 60 mg/kg Alloxan (Sigma)) on day −3. The level of blood glucose was monitored. A successful dog TID model was defined as its blood glucose level reach to 12 mM or higher in two consecutive days. The pharmaceutical composition was injected i.p on days 1, 4 and 7 as one treatment cycle. The second cycle was applied after two weeks later. Nine dogs with 15-20 kg were divided into 3 groups with 3 per group. The group 1 (100+15) was treated with 100 μg of rh-insulin plus 15 μg of DEX. Group 2 (100+1.5) was treated with 100 μg of rh-insulin plus 1.5 μg of DEX. Group 3 were diabetic group. The second cycle was applied two week later (Treated on day 21, 24, 27).

Detecting Levels of IL-10, TGF-β and Treg in Dogs.

1. Detection of IL-10, TGF-β in Dog.

The IL-10 and TGF-β expression were detected in pharmaceutical composition treated dogs on day-3, 0, 8, 20 and 28.

Specific procedures are: 1) The PBMCs from immunized dogs were isolated and purified by Ficoll, in which 4 ml of Ficoll400 (Sigma) were added under the 8 ml of dog blood and spin down at 1500 rpm for 15 minutes. The PBMC phase was taken out and washed after spin down. Then the PBMC were stimulated with 10 μg/ml of rh-insulin in vitro. 2) 24 hours later of stimulation, the stimulated cells were collected and Trizol added into the cell solution for RNA extraction. 3) The RNA were purified and transcribed into cDNA by Toyobo ReversTraAce kit according to the instruction. 4) The primers for HPRT, IL-10 and TGF-β amplification were designed as bellows:

HPRT p1: 5′-AGCTTGCTGGTGAAAAGGAC-3′
HPRT p2: 5′-TTATAGTCAAGGGCATATCC-3′
IL-10 p1: 5′-ATGCATGGCTCAGCACCGCT-3′
IL-10 p2: 5′-TGTTCTCCAGCACGTTTCAGA-3′
TGF-β p1: 5′-TGGAACTGGTGAAGCGGAAG-3′
TGF-β p2: 5′-TTGCGGAAGTCAATGTAGAGC-3′

The endogenous gene encoding the HPRT as a reference gene was amplified and adjusted to the same level for each sample. 5) The IL-10 and TGF-β were amplified by PCR and the expressions were analyzed by DNA gel running and Gelpro software. The dogs without diabetic symptom were set up as control group.

The result in FIG. 13 shows that composition treatment significantly up-regulate the level of IL-10 and TGF-β in dogPBMCs. However, less dose dependent effects were observed (FIG. 13).

2. Detection of Treg in Dogs.

The Treg populations were detected on day 2 after immunization. The percentage of Treg can reflect that composition treatment could induce Treg.

The pancreatic cells prepared 2 days after final immunization (day 28). The specific procedures are: 1) The dogs were sacrificed 100 ml 1 mg/ml of Collagenase P (Roche, Cat. No. 11213857001) were injected into pancreatic tissue and digested for 1 hour at 37° C. 2) The pancreatic cells were washed twice and spin down at 250 g for 1 minute. 3) The cell pellets were re-suspended with 15 ml of 25% Ficoll (Roche) and in turns 9 ml of 23% Ficoll, 6 ml of 20% Ficoll, 6 ml of 11% Ficoll were added into above the 23% Ficoll. Then spin down the cell solution at 800 g for 10 minutes and the islet cell phase were taken out, the Ficoll solution in islet cell was washed out with twice PBS washing. 4) The islets were digested with 0.25% of trypsin for for 10 minutes at 37° C. 5) The single suspension islet cells were stained with surface marker of CD4 (FITC-anti-CD4, eBioscience 11-5040) and intracellular protein of Foxp3 (PE-anti-Foxp3, eBioscience 12-5773), then the stained markers were analyzed by FACs

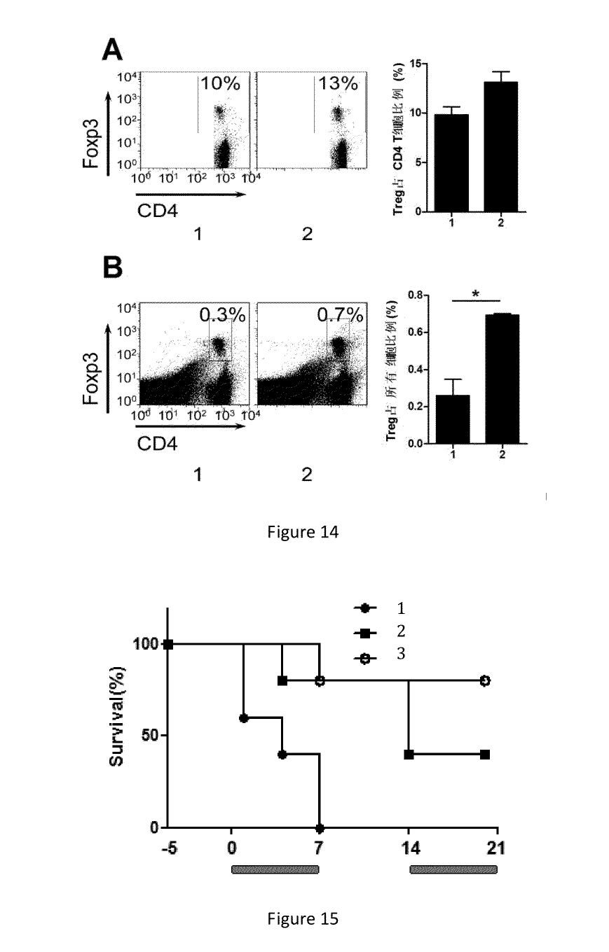

The result in FIG. 14 shows that the percentage of Treg to CD4 cells increased whereas the percentage of Treg to all cells significantly increased in group 2 (100+1.5).

### Example 6

### Evaluation of Treatment Effect on TID Dogs

The composition (rh-Insulin+Dex) and dosing were examined in the Example 5; the effect was further evaluated on composition of rh-insulin plus CsA.

Dog Diabetic Model Induction and Immunization.

Induction of dog type I diabetic model were according to the Example 5, 15 dogs with 15 kg body weight were injected with 60 mg/kg Alloxan (Sigma)) on day −5. The level of blood glucose was monitored. A successful dog TID model was defined as its blood glucose level reach to 12 mM or higher in two consecutive days. The dogs were divided into 3 groups at 5 per group and injected i.p on days 1, 4 and 7 as one treatment cycle. The group 1 were injected 100 ug rh-insulin (0.15 IU/kg body weight) and 15 ug of Dex (1 ug/kg body weight) in 100 ul of PBS; group 2 were injected with 100 ug rh-insulin and 1.5 mg of CsA (100 ug/kg body weight) in 100 ul of PBS; group 3 were injected with 100 ul of PBS only for the disease model. The second treatment cycle was applied one week later (day 21, 24, 27).

Changes of Survival, Level of Blood Glucose and Body Weights in Dogs.

1. Detection of Survival in Dogs

Dog survives will reflect quality of dog life and efficacy of the treatments. Survived dogs were counted and calculated after the treatments.

As showed in FIG. 15, all dogs dead in the group 3, 2 survived in the group 1 on day 21 (40% of survival), 3 survived in the group 2 on day 21 (60% survival). This evaluation demonstrated that the composition of rh-insulin plus Dex or rh-insulin plus CsA increased the chance survival for these TID dogs.

2. Detection of Level Change of Blood Glucose.

To examine changing level of blood glucose would reflect efficacy of the treatment.

Test procedure: 10 ul of blood samples were spotted on the test strips and the test instrument read the concentration of blood glucose.

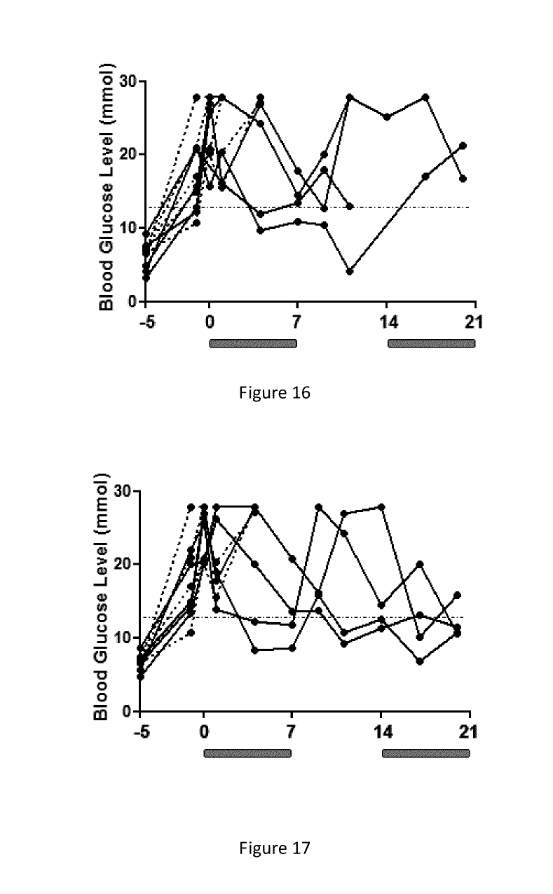

The result showed that level of blood glucose were relatively regulated in the group 1 with some of fluctuation (FIG. 16, indicated as the solid line); level of blood glucose were complete regulated in group 2 at range of 10-15 mmol (FIG. 17, indicated as the solid line), whereas, all animals dead in group 3 (FIGS. 16 and 17, indicated as the dashed lines).

3. Detection of Body Weight Change in Dogs

To examine changing body weights would reflect efficacy of the treatment.

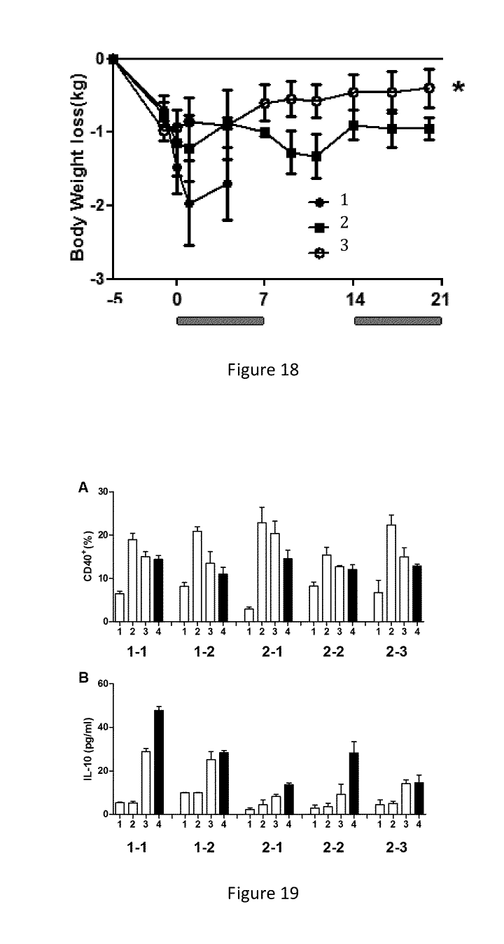

As showed in FIG. 18, reduction of body weight were control in some degree in group 1, completely control in group 2. Whereas group 3 all dead.

### Example 7

### The Pharmaceutical Composition Effects on Human DC Conversion from TID Patents' PBMC

The pharmaceutical composition effect was further evaluated on human DC conversion after human PBMCs treated with the pharmaceutical composition.

PBMC Collecting and Processing from Human Blood.

Three blood samples from normal human individuals and 2 blood samples from TID patients were collected at 10 ml for each. The PBMCs were isolated by Ficoll, in which 4 ml of Ficoll400 (Sigma) were added under the 8 ml of blood and spin down at 1500 rpm for 15 minutes. The PBMC phase was taken out and washed after spin down. Then the PBMC were stimulated with rhGM-CSF and rhIL-4 (R&D System) for 3 days. The induced DC were seeded into 96-well plate for 2×10⁶ cells per well and further stimulated with 1) 10 μg/ml of rh-insulin, 2) 10 μg/ml of DEX, 3) 10 μg/ml of rh-insulin plus DEX.

### Detection of DC Related Markers and Suppressive IL-10 Level

### 1. Detection of CD40, CD80, CD83, CD86, MHC-II on DCs.

The surface markers, CD40, CD80, CD83, CD86, MHC-II on DC, were detected after 3 days stimulation. The inhibition effect by pharmaceutical composition treatment on DC maturation was determined as in following protocols.

1) The DCs were stained with different cell marker combos, and listed as following, CD1a-FITC (eBioscience, 11-0019), CD40-PE (eBioscience, 12-0409), CD1a-FITC (eBioscience, 11-0019) and CD80-PE (eBioscience, 12-0809), CD1a-FITC (eBioscience, 11-0019), D83-PE (Biolegend, 305322), CD1a-FITC (eBioscience, 11-0019), CD86-PE (eBioscience, 12-0869), CD1a-FITC (eBioscience, 11-0019) and MHC-II-PE. In which, the 0.25111 of each antibody per 10⁶ cells was premixed and added. 2) The stained cells were detected by FACs after 10 minutes staining and washings.

The result in FIG. 19A shows that the CD40 expression was down-regulated on DC from TID patient's and normal human PBMCs after the pharmaceutical composition treatment. CD40 down regulation would indicate that decreased matured DC could convert nave T cells into Treg cells.

### 2, Detection of Suppressive IL-10 Level.

The IL-10 expression detected after 3 days stimulation.

The detail procedures are: 1) The 30 ul supernatant of cells were collected and mixed with 30 μl PBS which contained 0.1 μl FlexSet microbead for 30 minutes. The 30 μl PBS that contained 0.1 μl anti-IL-10 PE antibody were added and incubated for another 30 minutes. 2) The IL-10 expressions were measured by FACs.

The result in FIG. 19B shows that the IL-10 expressions were significantly up-regulated after the pharmaceutical composition treatment.

### Example 8

### The Screening of Immune-Suppressive Agents on Human PBMC to DC Conversions

The results of Example 6 indicate that DC can be induced from human PBMCs after composition treatment. The other immune suppressive agents were further screened to enhance the therapeutic effect of composition.

The immune-suppressive agents in this example are DEX, Rap, CsA and FK506.

Experimental materials; 25 blood samples from TID patients were collected at 10 ml of each sample. The PBMCs were isolated by Ficoll, in which 4 ml of Ficoll400 (Sigma) were added under the 8 ml of blood and spin down at 1500 rpm for 15 minutes. The PBMC phase was taken out and washed after spin down. Then the PBMC were stimulated with rhGM-CSF and rhIL-4 (R&D System) for 3 days. The induced DC were seeded into 96-well plate for 2×10⁶ cells per well and further stimulated with 1) 10 μg/ml of rh-insulin, 2) 10 μg/ml of rh-insulin plus DEX, 2) 10 μg/ml of rh-insulin plus Rap, 4) 10 μg/ml of rh-insulin plus CsA, 5) 10 μg/ml of rh-insulin plus FK506.

Detection of DC Related Markers and Suppressive IL-10 Level

1. Detection of CD40, CD80, CD83, CD86, MHC-II on DC.

The surface markers, CD40, CD80, CD83, CD86, MHC-II on DC were detected after 3 days stimulation. The effects of different pharmaceutical composition on DC maturation were determined as following protocols.
1) The stimulated DC were stained with different cell marker combo, CD1a-FITC (eBioscience, 11-0019) and CD40-PE (eBioscience, 12-0409), CD1a-FITC (eBioscience, 11-0019) and CD80-PE (eBioscience, 12-0809), CD1a-FITC (eBioscience, 11-0019) and D83-PE (Biolegend, 305322), CD1a-FITC (eBioscience, 11-0019) and CD86-PE (eBioscience, 12-0869), CD1a-FITC (eBioscience, 11-0019) and MHC-II-PE; In which, the 0.25 μl of each antibody per 10⁶ cells were added. 2) The stained cells were detected by FACs after 10 minutes staining and washing.

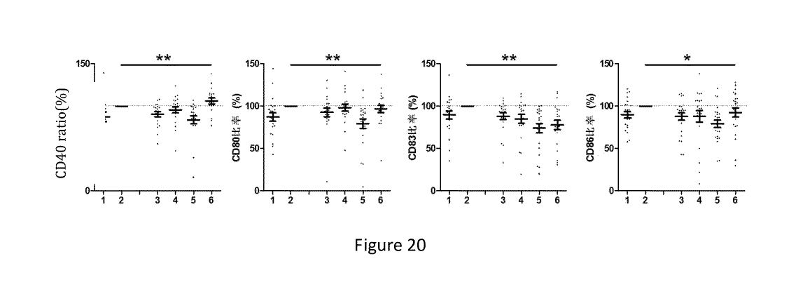

The result shows that the expressions of CD40, CD80, CD80 and CD86 were down regulated after the pharmaceutical composition treatment. The most effective pharmaceutical compositions are the DEX and CsA in comparisons. (FIG. 20)

### INDUSTRY APPLICATION

The present invention provides a composition for treating and/or preventing Type I can increase the ratio of CD4⁺CD25⁺Treg to CD4⁺T cells; enhance the proliferation of CD4⁺CD25⁺ Treg; increase the IL-10 secretion in T cells; control the blood glucose; inhibit the cytotoxicity effect of auto-reactive CD8⁺T cells; up regulate the transcription level of IL-10 or/and TGF-β in PBMC or/and spleen cells; inhibit the DC maturation; to induce immune suppression, as the consequence, the TID can be effectively cured.

1-11. (canceled)

12. A composition capable of eliciting a suppressive immune response against type 1 diabetes (TID), comprising an antigen and immunosuppressive agent.

13. The composition of claim 12, wherein the antigen is a protein antigen.

14. The composition of claim 12, wherein the antigen is a peptide antigen.

15. The composition of claim 12, comprising a mixture of a protein antigen and a peptide antigen.

16. The composition of claim 13, wherein the protein antigen is selected from the group consisting of Insulin, Glutamic Acid Decarboxylase (GAD65) and Islet Amyloid Polypeptide (IAPP), or a fragment thereof.

17. The composition of claim 12, wherein the immunosuppressive agent is selected from the group consisting of Dexamethasone (Dex), tacrolimus (FK506), cyclosporine (CsA), mycophenolate mofetil (MMF), azathiopurine (Aza), prednisone (Pred), methylprednisolone (MP), and a monoclonal antibody against CD3 or CD4, or a combination thereof.

18. The composition of claim 14, wherein the peptide antigen comprises at least one epitopic sequence from insulin, GAD65, IAPP, or a combination thereof.

19. The composition of claim 12, wherein the antigen is from human, dog or cat.

20. The composition of claim 12, wherein the antigen is a native protein or peptide.

21. The composition of claim 12, wherein the antigen is chemically synthesized.

22. The composition of claim 18, wherein the epitopic sequence from insulin comprises an amino acid sequence set forth in SEQ ID NOS: 1, 2, 3, 4, 5, 6, 7, 12, or 13.

23. The composition of claim 18, wherein the epitopic sequence from human GAD65 comprises an amino acid sequence set forth in SEQ ID NO: 8.

24. The composition of claim 18, wherein the epitopic sequence from human IAPP comprises an amino acid sequence set forth in SEQ ID NO: 9.

25. The composition of claim 18, wherein the epitopic sequence from dog IAPP comprises an amino acid sequence set forth in SEQ ID NO: 10.

26. The composition of claim 18, wherein the epitopic sequence from cat IAPP comprises an amino acid sequence set forth in SEQ ID NO: 11.

27. The composition of claim 12, wherein the weight ratio of the antigen to the immunosuppressive agent is about 1:20 to about 20:1.

28. A method of vaccination comprising administering the composition of claim 12 to a subject in need thereof.

29. The method of claim 28, wherein the administering route is selected from the group consisting of intramuscular, intra subcutaneous, and intradermal.

30. The method of claim 28, wherein one or more of the functions listed below are elicited:
(1) Treatment and/or prevention of TID;
(2) Enhance the proliferation of CD4⁺ CD25⁺Treg population;
(3) Increase the ratio of CD4⁺CD25⁺Treg to CD4⁺T cells;
(4) Increase the IL-10 secretion in T cells;
(5) Inhibit the cytotoxicity effect of auto reactive CD8⁺T cells;
(6) Control the blood glucose for TID patient;
(7) Up-regulate the transcription level of IL-10 and/or TGF-β in PBMC and/or spleen cells;
(8) Inhibit the DC maturation; and
(9) Decrease the expression of at least one protein of CD40, CD80, CD83, or CD86 in DC.


SEQUENCE LISTING (13 sequences)

SEQ ID NO: 1 - protein, 15 aa, Artificial Sequence
  Synthetic
SHLVEALYLVCGERG

SEQ ID NO: 2 - protein, 9 aa, Artificial Sequence
  Synthetic
LYLVCGERG

SEQ ID NO: 3 - protein, 31 aa, Artificial Sequence
  Synthetic
EAEDLQVGQVELGGGPGASGLQPLALEGSLQ

SEQ ID NO: 4 - protein, 15 aa, Artificial Sequence
  Synthetic
SHLVEALYLVCGERG

SEQ ID NO: 5 - protein, 9 aa, Artificial Sequence
  Synthetic
LYLVCGERG

SEQ ID NO: 6 - protein, 15 aa, Artificial Sequence
  Synthetic
SHLVEALYLVCGERG

SEQ ID NO: 7 - protein, 9 aa, Artificial Sequence
  Synthetic
LYLVCGERG

SEQ ID NO: 8 - protein, 10 aa, Artificial Sequence
  Synthetic
VMNILLQYVV

SEQ ID NO: 9 - protein, 37 aa, Artificial Sequence [sequence omitted]

SEQ ID NO: 10 - protein, 37 aa, Artificial Sequence
  Synthetic
KCNTATCATQRLANFLVRTSNNLGAILSPTNVGSNTY

SEQ ID NO: 11 - protein, 37 aa, Artificial Sequence
  Synthetic
KCNTATCATQRLANFLIRSSNNLGAILSPTNVGSNTY

SEQ ID NO: 12 - protein, 17 aa, Artificial Sequence
  Synthetic
GFFYTPKTRREAEDLQV

SEQ ID NO: 13 - protein, 27 aa, Artificial Sequence
  Synthetic
HLVEALYLVCGERGFFYTPKTRREAED